(19) United States
(12) Patent Application Publication
(10) Pub. No.: US 2016/0058398 A1
(43) Pub. Date: Mar. 3, 2016

(54) ROTOR FOR A GANTRY OF A COMPUTED TOMOGRAPY APPARATUS

(71) Applicant: SIEMENS AKTIENGESELLSCHAFT, Muenchen (DE)

(72) Inventor: Aurel Jensch, Eggolsheim (DE)

(73) Assignee: SIEMENS AKTIENGESELLSCHAFT, Muenchen (DE)

(21) Appl. No.: 14/838,630

(22) Filed: Aug. 28, 2015

(30) Foreign Application Priority Data
Aug. 29, 2014 (DE) 102014217275.6

(51) Int. Cl. A61B 6/03; A61B 6/00

(52) U.S. Cl. CPC A61B 6/035; A61B 6/4435

(57) ABSTRACT

A rotor for a gantry of a computed tomography device is formed as a hollow body at least in one section. The hollow body has a hollow profile cross-section in at least one half-plane defined by the axis of rotation of the rotor.

### BACKGROUND OF THE INVENTION

1. Field of the Invention

The invention concerns a rotor for a gantry of a computed tomography apparatus.

2. Description of the Prior Art

Computed tomography apparatuses enable the reconstruction of three-dimensional layer or volume images of an examination region for diagnostic purposes. The reconstruction of an image is done on the basis of projections of the examination region that are obtained from different projection directions, by rotating an imaging apparatus so that measurement data for parallel projections are obtained from an angular range of at least 180 degrees, plus fan angle. In order to accomplish the rotation of the imaging apparatus, the computed tomography device has a gantry, which includes a stationary (non-rotating) pivoting frame and a rotor rotatably mounted in the frame bearing apparatus.

The purpose of the gantry of a computed tomography device is the guidance of components of an imaging apparatus on a path around the patient. This path is a circular path and is traversed repeatedly with high precision. The rotor, which can be embodied for instance as a drum or as an annular disk, forms a supporting structure that is suited for mounting the components of the imaging apparatus so as to produce a geometrically fixed situating of the components to the imaging apparatus. For instance, such a rotor can have a rotor wall in the form of an annular disk and a mounting ring circulating around the outer periphery of the rotor wall for mounting the components of the imaging apparatus. The rotor is conventionally manufactured as a cast part made of aluminum casting or an aluminum alloy, for instance AlZn10SiMg. The wall thicknesses of such a rotor vary between 15 and 20 mm.

A further purpose of the rotor is to assist with cooling of the rotating components, in particular the radiation source. The cooling can be implemented for example by cooling surfaces, by a coolant, or by a heat accumulator, which may be solid aluminum cast parts.

To prevent motion artifacts in the reconstructed image, which may occur due to patient or organ movements, attempts are made to select the time window for acquiring the projections required for reconstruction purposes to be as small as possible, by selecting high rotation speeds. Rotational speeds of 210 rpm are achieved with current computed tomography apparatuses.

By combining a high rotational speed, a large rotational radius, and a high rotational mass, the rotor represents a mechanically highly-stressed component, which, aside from absorbing the occurring stresses, must also guarantee that the positions of the x-ray tube and the detector are maintained, since position displacements of the components of more than 0.15 mm may already result in a significant impairment of the image quality.

Significant basic requirements placed on the rotor of a gantry are not only a high resistance when transmitting forces, but also a high rigidity in order to keep deformations of the rotor, and thus position displacements of the components of the imaging apparatus, below the permissible limits, with a minimum weight.

DE 10 2008 036 015 B4 discloses a rotor for a computed tomography device, which is manufactured, at least in sections, in a separate frame construction from beam-type base elements.

### SUMMARY OF THE INVENTION

An object of the present invention is to provide a rotor of a gantry of a computed tomography apparatus that has a high rigidity and a high resistance and a low weight, such that high rotor rotational speeds can be realized without impairing the image quality of the generated images. An object of the invention is, moreover, to provide a gantry and a computed tomography device with such a rotor.

The inventive rotor for a gantry of a computed tomography apparatus formed as a hollow body at least in one section, with the hollow body having a hollow profile cross-section in at least one half-plane defined by the axis of rotation of the rotor. The hollow body expediently forms a supporting structure, which is suited to mounting components of a computed tomography device, in particular components of an imaging apparatus.

A solid absorbs mechanical stresses, particularly with bending moments, primarily in a planar region directly below the surface of the solid, which has a minimal wall thickness in relation to the extent of the solid. In relation to the planar region, the middle “neutral fiber” contributes minimally to the rigidity and resistance. The arrangement of material to form a hollow body achieves a higher rigidity and resistance compared with a solid with the same material usage. Similarly the material usage can be reduced in this way with the remaining resistance. The hollow body is preferably formed such that material is used precisely where it is required to receive the stresses produced during rotation of the rotor. The rotor embodied as a hollow body is thus characterized by a low material usage and thus by a low overall weight of the rotor compared with a solid rotor.

Because the achieved reduction in the weight and the rotational mass associated with the weight, which has to be accelerated during rotation of the rotor, higher rotational speeds of the rotor can be realized with a comparatively lower dimensioning of the drive. The transport costs for the rotor are also significantly reduced due to the weight reduction.

The hollow body preferably has the form of at least one segment of a ring proceeding around the axis of rotation of the rotor. A hollow body that has a hollow profile cross-section in at least one half-plane defined by the axis of rotation of the rotor, can be realized in a structurally simple manner in this form. In such cases the hollow profile cross-section can be open or closed. A closed hollow profile cross-section may be essentially circular, triangular or square in shape for instance. An open hollow profile cross-section may be essentially U-shaped, V-shaped or Ω-shaped, for instance. A particularly high rigidity and resistance of the rotor can be achieved by an essentially triangular hollow profile cross-section of the hollow body. With a triangular hollow profile cross-section, the walls of the hollow body are essentially only loaded under tension and pressure, as a result of which a particularly minimal wall thickness of the rotor can be realized. The hollow body preferably has a planar outer surface. Components of the computed tomography device can be arranged particularly easily on the planar outer surface. In an embodiment of the invention, the rotor has a rotor wall in the form of an annular disk. Components of the computed tomography device can be arranged particularly easily on the annular disk. The rotor wall preferably forms a section of the hollow body, at least in one section. The rotor wall expediently forms a planar outer surface of the hollow body at least in one section.

The rotor preferably has a reinforcing structure. The reinforcing structure has the form of at least one segment of a ring proceeding around the axis of rotation of the rotor. The ring has a hollow profile cross-section that is open in the direction of the rotor wall, and the reinforcing structure is connected with the rotor wall on the inner periphery and outer periphery of the rotor wall, such that a hollow body is formed. A hollow body having a hollow profile cross-section in at least one half-plane defined by the axis of rotation of the rotor can thus be realized in a structurally simple manner.

The inventive rotor is characterized in particular by the combination of a supporting structure and a chamber or cavity partition. As a result, components of the computed tomography device, for instance a cooling system, can be integrated on the rotor with minimal material and space outlay. In an embodiment of the invention, at least one cavity is embodied in the hollow body. A coolant guide is integrated in the hollow body for further efficient material savings. By integrating a fan in the hollow body, additional cooling ducts and complex air seals are avoided between the rotating and stationary part. The susceptibility to faults of the cooling facility is as a result reduced.

In another embodiment of the invention, the rotor has at least one planar part, wherein the at least one planar part forms a section of the hollow body at least in one section. The at least one planar part preferably has a very minimal wall thickness compared with the extension. The at least one planar part expediently imparts the hollow body with rigidity and resistance.

In another embodiment of the invention, the at least one planar part has a curvature around an axis that is at least approximately parallel to the axis of rotation of the rotor at least in one section, in which it forms a section of the hollow body. On account of a curvature about an axis which is at least approximately parallel to the axis of rotation of the rotor, the rigidity and the resistance of the at least one planar part and thus the rigidity and resistance of the hollow body, and thus the rigidity and the resistance of the rotor, are advantageously increased. A hollow body that has a hollow body comprises a hollow profile cross-section in at least one half-plane defined by the axis of rotation of the rotor, can be realized in a structurally easy manner with this feature. It is also possible for the at least one planar part to have a curvature in two spatial directions at least in one section, in which it forms a section of the hollow body, as a result of which the rigidity and resistance of the rotor are additionally increased.

The curvature of the at least one planar part is preferably locally adjusted to stress curves that occur during rotation of the rotor, so that the load of the at least one planar part on account of bending and torsion stresses is reduced, as a result of which the at least one planar part is essentially loaded under tension and pressure. With the consistently high rigidity and resistance of the rotor, an at least planar part can be realized in this way with a comparatively minimal wall thickness, which significantly reduces the material usage and the overall weight of the rotor.

The rotor preferably has an inner ring for a ball bearing. The at least one planar part preferably forms a section of the inner ring at least in one section. The at least one planar part preferably comprises a wavy structure circulating in the peripheral direction of the inner ring at least in one section in which it forms a section of the inner ring. The at least one planar part forms a section of a pulley for the drive of the rotor at least in one section. The at least one planar part preferably forms a section of a flange for mounting a slip ring at least in one section. Additional components and links are avoided in this way.

The hollow body can be manufactured from a sheet metal, at least in one section. The hollow body is preferably manufactured at least in one section by drawing. The low material usage allows for the use of material comprising a higher mass density compared with aluminum alloy. The hollow body is preferably manufactured from sheet steel at least in one section. Material costs are significantly reduced as a result. The sheet steel is preferably strain hardened. A rotor with a high rigidity and resistance can thus be realized with a comparatively cost-effective material.

### BRIEF DESCRIPTION OF THE DRAWINGS

FIG. 1 is a perspective view of a part of a gantry of a computed tomography device having an inventive rotor and a stationary pivoting frame.

FIG. 2 is a further perspective view of the inventive rotor in FIG. 1.

FIG. 3 is a perspective view of a portion of an exemplary embodiment of a planar part.

FIG. 4 is a perspective view of a portion of a second exemplary embodiment of a planar part.

FIG. 5 is a sectional view of the inventive rotor along the lines V-V in FIG. 2.

FIG. 6 is a perspective view of an inventive rotor with a cooling system integrated into the rotor.

FIG. 7 is a sectional view through a part of a gantry of a computed tomography device having an inventive rotor and a stationary pivoting frame along the line VII-VII in FIG. 1.

FIG. 8 is a top view of a cutout in the axial direction through an inventive rotor with an integrated pulley for the drive of the rotor

FIG. 9 is a top view of a cutout in the axial direction through an inventive rotor with an integrated flange and a slip ring fixed to the flange.

FIG. 10 is a perspective view of a computed tomography apparatus of the type in which the inventive rotor can be used.

### DESCRIPTION OF THE PREFERRED EMBODIMENTS

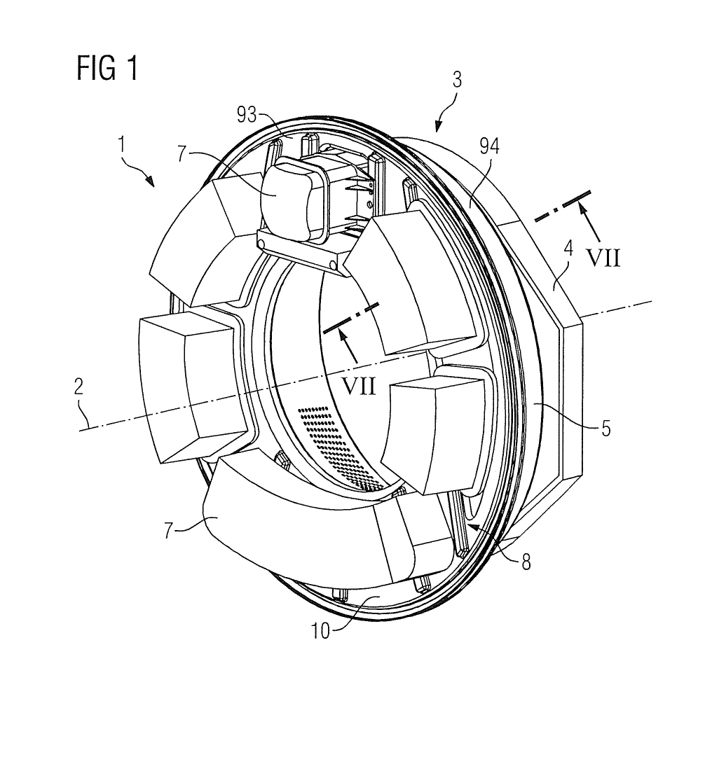

A part of a gantry 3 of a computed tomography device 27 is shown in FIG. 1 in a perspective view, wherein the gantry 3 includes a stationary (i.e., non-rotating, but pivoting) frame 4 and an inventive rotor 1 arranged so as to be rotatable about a pivot bearing apparatus 6. The rotor 1 is embodied as a hollow body 8 at least in one section. The hollow body 8 has a hollow profile cross-section in at least one half-plane defined by an axis of rotation 2 of the rotor 1. One example of a half-plane defined by the axis of rotation 2 of the rotor 1 is a half-plane which is limited by the axis of rotation 2 of the rotor 1 in FIG. 1 and includes the line of intersection VII-VII.

The hollow body 8 preferably forms a supporting structure, which is suited to mounting a component 7 of an imaging apparatus of the computed tomography device 27. The rotor 1 preferably has at least one planar part 5 that forms a portion of the hollow body 8, at least in one angle segment around the axis 2. The at least one planar part 5 preferably has a curvature about an axis which is at least approximately parallel to the axis of rotation 2 of the rotor 1 at least in one section. Here the curvature has a curvature radius between 10 millimeters and 10 meters.

In an embodiment of the invention, the hollow body 8 has the form of at least one segment of a ring proceeding around the axis of rotation 2 of the rotor 1. The hollow body 8 has the form of ring proceeding entirely around the axis of rotation 2 of the rotor 1.

The rotor 1 has a rotor wall 93 in the form of an annular disk. The rotor wall 93 preferably has a planar outer surface 10. Components of the computed tomography device 27, for instance components 7 of the imaging apparatus, can be arranged particularly easily on the planar outer surface 10 for instance by means of spot bonding. The rotor 1 preferably has at least one planar part 5 that forms a portion of the rotor wall 93 at least in one angular segment around the axis 2. The rotor wall 93 is preferably manufactured in one portion from sheet metal. The rotor wall 93 is preferably manufactured at least in one portion by drawing. The rotor wall 93 is preferably manufactured from steel sheet at least in one portion. The sheet steel is preferably strain hardened. The rotor wall 93 thus can be realized with a comparatively minimal wall strength, as a result of which material usage is reduced.

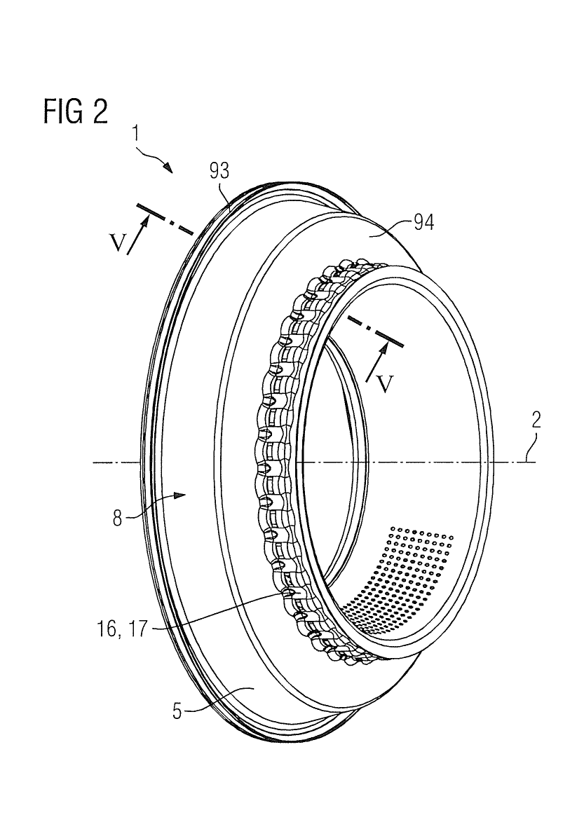

The inventive rotor 1 of FIG. 1 is shown in FIG. 2 in a further perspective view. The rotor 1 has a reinforcing structure 94 in the form of at least one angular segment of a ring proceeding around the axis of rotation 2 of the rotor 1. The ring has a hollow profile cross-section that is open in the direction of the rotor wall 93. The reinforcing structure 94 is connected with the rotor wall 93 on the inner periphery and on the outer periphery of the rotor wall 93 such that the hollow body 8 is formed. The hollow body 8 formed in this way has a hollow profile cross-section in a half-plane defined by the axis of rotation 2 of the rotor 1. One example of a half-plane defined by the axis of rotation 2 of the rotor 1 is a half-plane that is limited by the axis of rotation 2 of the rotor 1 in FIG. 2 and includes the line V-V. The reinforcing structure 94 preferably has an essentially V-shaped hollow profile cross-section. The hollow body 8 thus expediently comprises an essentially triangular hollow profile cross-section. An essentially triangular hollow profile cross-section provides the hollow body 8 with a particularly high rigidity and resistance.

The rotor 1 preferably has at least one planar part 5 that forms a portion of the reinforcing structure 94 at least in one angular segment around the axis 2. The planar part 5 has a curvature along two axes essentially at right angles to one another, at least in the portion in which it forms the segment of the reinforcing structure 94. One of the axes is preferably approximately parallel to the axis of rotation 2 of the rotor 1. The reinforcing structure 94 is manufactured at least in one portion from sheet metal. The reinforcing structure 94 is preferably manufactured at least in one section by drawing. The sheet steel is preferably strain hardened here. A reinforcing structure 94 can thus preferably be realized with a comparatively minimal wall strength, as a result of which material usage is reduced.

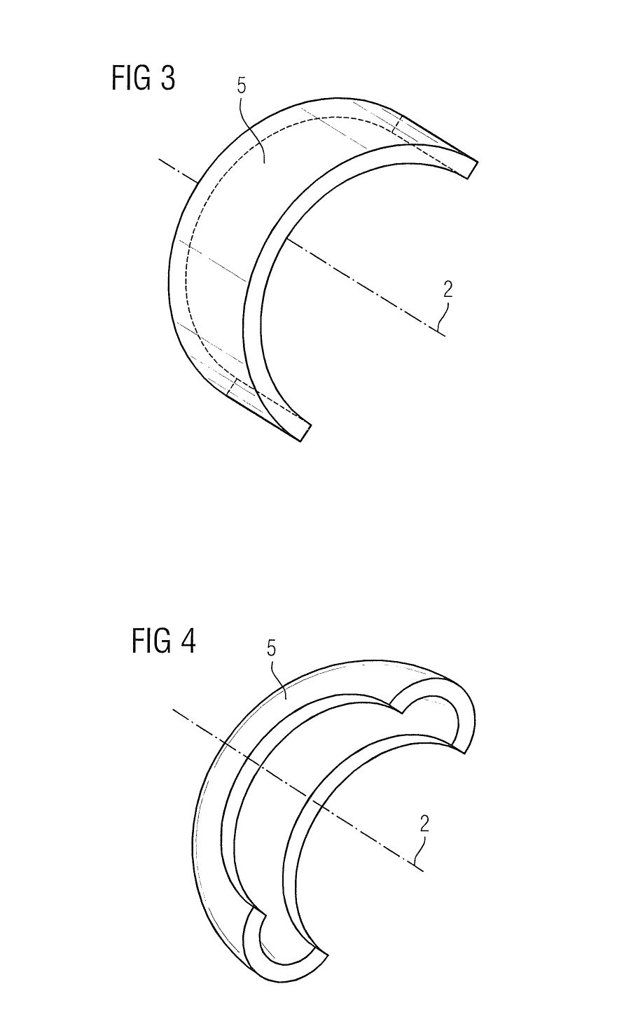

FIG. 3 shows a perspective view of a portion of the part 5 that has a curvature around an axis which is at least approximately parallel to the axis of rotation 2 of the rotor at least in one portion.

FIG. 4 shows a perspective view of a portion of the part 5 that has a curvature around two axes that are essentially at right angles to one another at least in one section. One of the two axes is preferably approximately parallel to the axis of rotation 2 of the rotor 1. A curvature, in particular a curvature around two axes that are essentially at right angles to one another, provides the at least one planar part 5 with particularly high rigidity and resistance.

The at least one planar part 5 may have different curvatures. It makes sense here for the curvature of the at least one planar part 5 to be locally adjusted as a function of voltage curves produced during the rotation of the rotor 1. It is possible in this way for the at least one planar part 5 to be adapted to the local requirements with respect to mechanical load.

By selecting an appropriate curvature and support of the at least one planar part 5, it is possible for the stresses occurring in the rotor 1 to essentially be absorbed by tension and pressure stresses that are present in the at least one planar part 5, so the bending and torsion stresses in the at least one planar part 5 are minimized. The at least one planar part 5 is expediently aligned at least in one section such that the force paths produced during rotation of the rotor 1 proceed tangentially with respect to the at least one planar part 5. The forces are introduced tangentially on the support of the at least one planar part 5. The planar part 5 thus can absorb and transfer the forces produced in the rotor 1 and the stresses associated therewith in an improved manner.

The rigidity and resistance of the rotor 1 is in this way essentially determined by the curvature and support of the at least one planar part 5 and only to a minimum degree by the wall thickness of the at least one planar part 5. Very minimal wall thicknesses of the at least one planar part 5 can thus be realized when maintaining the rigidity and resistance of the rotor 1. A significant reduction in the weight or rotational mass can be effected as a result. In this way high rotor rotational speeds can be realized while maintaining the rigidity and the resistance of the rotor 1 with comparatively small dimensioning of a drive of the rotor 1.

Spatial stress curves and the curvature and support of the at least one planar part 5 required to minimize the bending and torsion stresses may be determined experimentally or numerically in the form of a simulation using corresponding mathematical models.

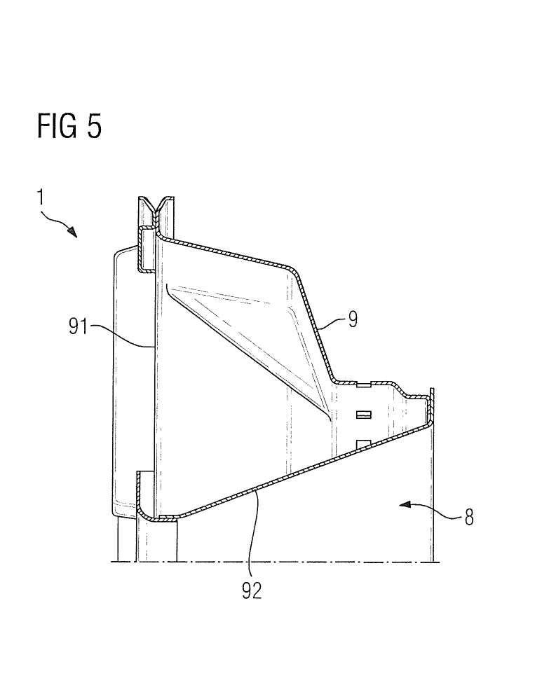

FIG. 5 is a sectional view of the inventive rotor 1 along the line V-V in FIG. 2. The hollow body 8 has a hollow profile cross-section in at least one half-plane defined by the axis of rotation 2 of the rotor 1. For instance, the hollow body 8 has a hollow profile cross-section in a half-plane defined by the axis of rotation 2 of the rotor 1, the half-plane including the line V-V. As an example, FIG. 5 shows a hollow profile cross-section in at least one half-plane defined by the axis of rotation 2 of the rotor 1. The hollow profile cross-section is preferably essentially triangular here. Here the hollow body 8 has a first wall 9, a second wall 91 and a third wall 92, wherein the three walls are arranged in a triangular manner. Here at least one wall 9, 91 or 92 can be arched toward the outside and/or the inside of the hollow body 8 at least in one section. Moreover, at least one wall 9, 91 or 92 can comprise structures for reinforcement, for instance crimping, or holding means for components. Two of the three walls 9, 91, 92, preferably the wall 9 and the wall 92, preferably form at least one section of a reinforcing structure 94. One of the three walls 9, 91, 92, preferably the wall 91, preferably forms at least one section of a rotor wall 93. A load on the at least one wall 9, 91 or 92 on account of bending stresses can be particularly effectively minimized by an essentially triangular hollow profile cross-section. The at least one planar part 5 preferably forms a section of at least one of the three walls 9, 91, 92 in at least one portion. A load on account of bending stresses of the at least one planar part 5 can thus also be minimized particularly effectively. It would also be conceivable however for the hollow profile cross-section to be square, circular or open for instance. Expediently the hollow profile cross-section is essentially convex. With a convex hollow profile cross-section, each internal angle, which is formed by a pair of walls of the hollow body 8, is smaller than or equal to 180 degrees.

The hollow body 8 preferably forms a supporting structure. At the same time the hollow body can form a chamber partition. As a result, at least one cavity 11 can be embodied on the rotor 1 with little outlay in terms of space and material.

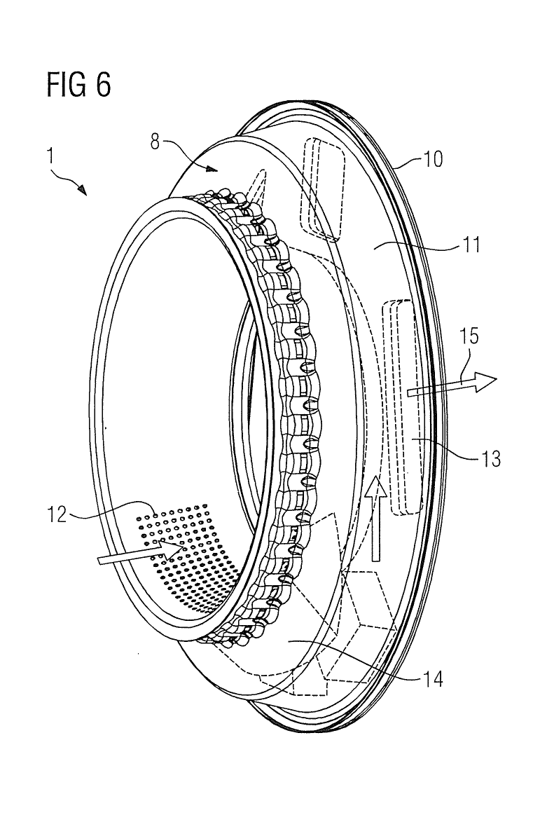

FIG. 6 shows a perspective view of an inventive rotor 1, wherein at least one cavity 11 is embodied in the hollow body 8 at least in one section. It would naturally also be conceivable to embody more than one cavity 11 in the hollow body 8. The cavities 11 can also be used for different purposes, for instance to supply or draw off coolant or to guide electronic or optical lines. In order to dampen vibrations, it would be conceivable for at least one part of the hollow body 8 to have a filler with vibration-damping properties.

In an embodiment of the invention, the hollow body 8 has a first opening 12, via which a coolant flows into the cavity 11 during operation. The hollow body 8 also has a second opening 13, by way of which the coolant flows out of the cavity 11 during operation.

Components 7 of the imaging apparatus that are held on the rotor 1 can thus be easily structurally cooled with little material usage. The coolant is preferably air, wherein a fan 14 is provided in the cavity 11, which generates an air flow 15 during operation, which flows into the cavity 11 via the first opening 12 and out of the cavity 11 via the second opening 13. The second opening 13 is preferably provided in a section of the hollow body that has a planar outer surface 10. Components 7 of the imaging apparatus can thus be easily arranged on the planar outer surface 10 and cooled by the outflowing airflow 15. By integrating the cooling system on the rotor 1, expensive air seals are avoided between the rotating and stationary part, as a result of which the susceptibility to faults of the cooling facility is reduced.

Mechanical components can be easily structurally integrated on the rotor 1 on account of a corresponding molding of the at least one planar part 5. As a result additional components and links are avoided, as a result of which manufacturing costs and susceptibility to faults are reduced. Moreover, subsequent machine processing steps of the planar part 5 for applying support elements can in this way be prevented, as a result of which the wall thickness of the planar part 5, and thus material usage are reduced.

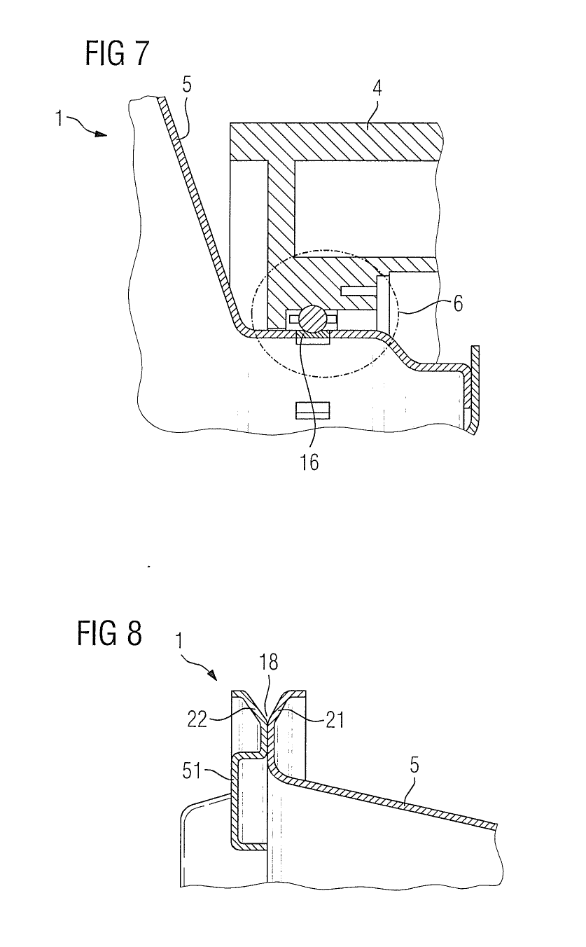

FIG. 7 is a sectional view of a part of a gantry 3 of a computed tomography device 27 with an inventive rotor 1 and a stationary pivoting frame 4 along the line VII-VII in FIG. 1, wherein the rotor 1 is rotatably mounted in a ball bearing 6. The rotor 1 has at least one planar part 5, wherein the planar part 5 forms a section of an inner ring 16 of a ball bearing 6 in at least one section. The at least one planar part 5 has a wavy structure 17 proceeding in the peripheral direction of the inner ring 16 in at least one section in which it forms a section of the inner ring 16. The at least one planar part 5 preferably has recesses in at least one section, in which it forms a section of the inner ring 16. A recess is preferably provided in each case between two adjacent waves of the wavy structure 17. The recesses are expediently dimensioned such that rolling elements of the pivot bearing apparatus 6 can be inserted into recesses in the radial direction leading to the axis of rotation 2 of the rotor 1.

FIG. 8 shows a top view of a part of a cutout in the axial direction through an inventive rotor 1, wherein the rotor 1 has at least one planar part 5. At least one section of the at least one planar part 5 forms at least one section of a pulley 18 for the drive of the rotor 3. The rotor 1 preferably has a first planar part 5 and a second planar part 51, which are joined to one another along at least one segment of their annular periphery, such that a lip 21 of the first planar part 5, protruding beyond the joining point, and a lip 22 of the second planar part 51, protruding beyond the joining point, form at least one section of a pulley 18. It would naturally also be conceivable to form the pulley 18 at least in one section by a groove-type depression in the planar part 5.

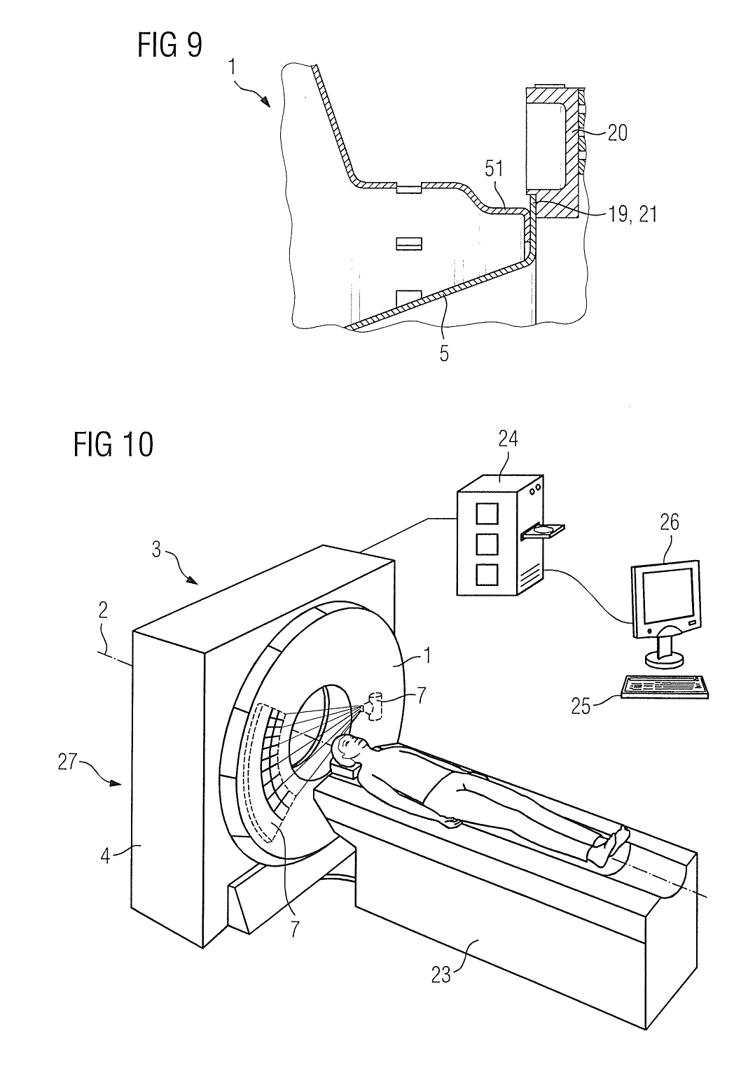

FIG. 9 is a top view of a part of a cutout in the axial direction through an inventive rotor 1, wherein the rotor 1 has at least one planar part 5, wherein the planar part 5 forms a section of a flange 19 for support of a slip ring 20 at least in one section. The rotor 1 preferably has a first planar part 5 and a second planar part 51, which are joined to one another along at least one segment of their annular periphery, such that a lip 21 of the first planar part 5, protruding beyond the joining point, forms at least one section of the flange 19. The slip ring 20 is preferably arranged on the flange 19 in a structurally simple manner, for instance by spot bonding.

The at least one planar part 5 is expediently manufactured at least in one section from sheet metal. It would likewise be conceivable to manufacture the planar part 5 from aluminum, plastic or composite material. The at least one planar part 5 is preferably manufactured at least in one section by drawing. Even complex curvatures and complex forms of the at least one planar part 5 can be manufactured mechanically with little outlay by drawing. The low material usage allows for the use of material having a higher mass density compared with aluminum alloy. The at least one planar part 5 is preferably manufactured from sheet steel at least in one section. Material costs are as a result significantly reduced. The sheet steel is preferably strain hardened. On account of the high resistance of strain hardened sheet steel, particularly thin wall thicknesses of the at least one planar part 5 can be realized. A rotor 1 with a high rigidity and resistance can thus be realized with a comparatively cost-effective material. The sheet metal has a thickness of 7 mm or less, such as the range 0.1 to 6 mm, preferably 0.5 to 5 mm, more preferably 1 to 4.5 mm.

FIG. 10 shows a perspective view of a computed tomography de-vice 27 having a gantry 3. The rotor 1 is expediently connected by the ball bearing 6, for instance a roller bearing, to a stationary pivoting frame 4, so that the rotor is rotatably mounted around an axis of rotation 2 that preferably is horizontal when the frame 4 is not pivoted, i.e., when the frame 4 is vertically oriented. Components 7 of the imaging apparatus 2 are mounted on the rotor 1. Moreover, the computed tomography device 2 has a patient bed 23, a computer 24, an input interface 25 and an output interface 26.

Although modifications and changes may be suggested by those skilled in the art, it is the intention of the inventor to embody within the patent warranted hereon all changes and modifications as reasonably and properly come within the scope of the inventor's contribution to the art.

I claim as my invention:

1. A rotor for a gantry of a computed tomography apparatus, said rotor comprising:
a rotor body configured to be received in a stationary frame of a gantry of a computed tomography apparatus and being rotatable within said frame around an axis of rotation; and
said rotor body having at least one section thereof formed as a hollow body, said hollow body having a hollow profile cross-section in at least one-half plane defined by said axis of rotation.

2. A rotor as claimed in claim 1 wherein said hollow body forms a segment of an annulus proceeding around said axis of rotation.

3. A rotor as claimed in claim 1 wherein said hollow profile cross-section of said hollow body is substantially closed.

4. A rotor as claimed in claim 1 wherein said hollow profile cross-section of said hollow body is substantially triangular.

5. A rotor as claimed in claim 1 wherein said hollow body comprises a planar outer surface, in at least one portion of said hollow body.

6. A rotor as claimed in claim 1 wherein said rotor has a rotor wall formed as an annular disk, and wherein at least one portion of said rotor wall forms a portion of said hollow body.

7. A rotor as claimed in claim 6 wherein said rotor body comprises a reinforcing structure, said reinforcing structure comprising at least a segment of an annular proceeding around said axis of rotation, said annulus having a hollow profile cross-section that is open in the direction of said rotor wall, and wherein said reinforcing structure is connected to said rotor wall at an inner periphery and at an outer periphery of said rotor wall, thereby forming said hollow body.

8. A rotor as claimed in claim 1 wherein said hollow body comprises at least one cavity therein, and wherein said hollow body comprises a first opening via which a coolant flows into said cavity, and a second opening via which said coolant flows out of said cavity.

9. A rotor as claimed in claim 8 comprising a fan in said cavity, said fan being configured to produce an air flow that causes to flow into said cavity via said first opening and air to flow out of said cavity via said second opening.

10. A rotor as claimed in claim 1 comprising at least one planar part forming a portion of said hollow body.

11. A rotor as claimed in claim 10 wherein said at least one planar part has a curvature around an axis that is substantially parallel to said axis of rotation.

12. A rotor as claimed in claim 10 wherein said at least one planar part has a curvature around two axes respectively at right angles to each other.

13. A rotor as claimed in claim 10 wherein said at least one planar part forms a portion of an inner ring of a bearing for said rotor body, said bearing allowing said rotor body to rotate within said stationary frame.

14. A rotor as claimed in claim 13 wherein said at least one planar part has a wavy structure proceeding in a peripheral direction of said inner ring, and forming a portion of said inner ring.

15. A rotor as claimed in claim 10 wherein said at least one planar part forms a portion of a pulley for drive of said rotor.

16. A rotor as claimed in claim 10 wherein said at least one planar part forms a section of a flange that supports a slip ring.

17. A rotor as claimed in claim 1 wherein said at least planar part is formed from sheet metal.

18. A rotor as claimed in claim 17 wherein said sheet metal has a thickness of 7 mm or less.

19. A computed tomography apparatus comprising:
a gantry comprising a stationary frame and a rotor mounted in said stationary frame;
said rotor having a rotor body configured to be rotatable within said frame around an axis of rotation; and
said rotor body having at least one section thereof formed as a hollow body, said hollow body having a hollow profile cross-section in at least one-half plane defined by said axis of rotation.
